(19) United States
(12) Patent Application Publication
(10) Pub. No.: US 2007/0110805 A1
(43) Pub. Date: May 17, 2007

(54) Modified-release pharmaceutical compositions

(76) Inventors: R. Saul Levinson, Chesterfield, MO (US); Jonathan D. Bortz, St. Louis, MO (US); Elio Mariani, Chesterfield, MO (US); Daniel J. Thompson, Ballwin, MO (US)

Correspondence Address: HARNESS, DICKEY, & PIERCE, P.L.C, 7700 BONHOMME, STE 400, ST. LOUIS, MO, 63105, US

(21) Appl. No.: 11/430,269

(22) Filed: May 8, 2006

Related U.S. Application Data
(60) Provisional application No. 60/679,123, filed on May 9, 2005.

(51) Int. Cl. A61K 31/485; A61K 31/46; A61K 31/445; A61K 9/22

(52) U.S. Cl. 424/468; 514/282; 514/304; 514/317; 514/524

(57) ABSTRACT

This invention is directed generally to modified-release pharmaceutical compositions, and, more particularly, to modified-release anesthetic- or analgesic-comprising pharmaceutical compositions that are bioadherent to a vaginal cavity surface, vulva surface, or skin. This invention also is directed generally to methods for preparing such compositions, methods of treatment using such compositions, uses of such compositions to prepare medicaments, and kits comprising such compositions.

### PRIORITY CLAIM TO RELATED PATENT APPLICATIONS

This patent claims priority to U.S. Provisional Patent Application No. 60/679,123 (filed May 9, 2005). The entire text of the '123 application is incorporated by reference into this patent.

### FIELD OF THE INVENTION

This invention is directed generally to modified-release pharmaceutical compositions, and, more particularly, to modified-release anesthetic- and/or analgesic-comprising pharmaceutical compositions that are bioadherent to a vaginal cavity surface, vulva surface, or skin. This invention also is directed generally to methods for preparing such compositions, methods of treatment using such compositions, uses of such compositions to prepare medicaments, and kits comprising such compositions.

### BACKGROUND OF THE INVENTION

Vulvodynia is a chronic vulvar discomfort or pain characterized by burning, stinging, irritation, or rawness of the female genitalia in cases in which there is no infection of the vulva or vagina causing these symptoms. Burning sensations are the most common, but the type and severity of symptoms are highly individualized. Pain may be constant or intermittent, and localized or diffuse. Vulvodynia has been classified into two subtypes—dysesthetic vulvodynia (also referred to as generalized vulvar dysesthesia) and vulvar vestibulitis syndrome (also referred to as vulvar dysesthesia localized in the vestibule).

Dysesthetic vulvodynia symptoms may be diffuse or in different areas at different times. Pain may be present in, for example, the labia majora, labia minora, and/or the vestibule. Some women experience pain in the clitoris, mons pubis, perineum, and/or the inner thighs. The pain may be constant or intermittent. Symptoms are not necessarily caused by touch or pressure to the vulva (e.g., with intercourse or bicycle riding), but these activities often exacerbate the symptoms. Dysesthetic vulvodynia is more common in postmenopausal women or younger women with history of back injury.

Vulvar vestibulitis is a chronic burning discomfort in the vulva, and believed to have multiple causes. Women with vulvar vestibulitis syndrome typically have pain only in the vestibule, and only during or after touch or pressure is applied. Burning sensations are the most common symptom and may be experienced with, for example, some or all of the following: sexual intercourse, tampon insertion, gynecologic examination, bicycle riding, and wearing tight pants. In 1987, Eduard Friedrich established three criteria for diagnosing vulvar vestibulitis: severe pain on vestibular touch or attempted vaginal entry, tenderness to pressure localized within the vulvar vestibule, and physical findings confined to vestibular erythema of varying degrees.

Galask et al. (U.S. Pat. No. 5,888,523) discuss a method for treating pain associated with vulvodynia or vulvar vestibulitis by applying a topical cream containing nonsteroidal anti-inflammatory drugs. Nyrjesy et al. (U.S. Pat. No. 6,150,400) discuss a method for treating vulvar vestibulitis by applying a composition comprising a compound that inhibits the release of mediators from mast cells. Zolnoun et al. (Obstetrics & Gynecology 102(1):84-87 (2003)) discuss a method for treating vulvar vestibulitis by applying 5% lidocaine ointment.

Applicants are unaware of any cure for dysesthetic vulvodynia, vulvar vestibulitis, or vulvodynia. Thus, there continues to be a need for alternative compositions and methods of treatments that may alleviate the symptoms of those diseases, thereby providing partial or complete relief. This invention provides compositions and methods of treatment that generally address such a need.

### SUMMARY OF THE INVENTION

This invention is directed generally to modified-release pharmaceutical compositions, and, more particularly, to modified-release anesthetic- and/or analgesic-comprising pharmaceutical compositions that are bioadherent to a vaginal cavity surface, vulva surface, or skin, as well as methods for preparing such compositions, methods of treatment using such compositions, uses of such compositions to prepare medicaments, and kits comprising such compositions. The compositions and methods of treatment are particularly suitable for use with humans, but may be used with other animals, particularly mammals, such as non-human primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

Briefly, therefore, this invention, is directed, in part, to modified-release pharmaceutical compositions that are bioadherent to a vaginal cavity surface, vulva surface, or skin. The compositions comprise two phases (i.e., at least two phases are present in the compositions). One phase is a hydrophobic external phase. Another phase is an aqueous internal phase that is encased and/or dispersed within the external phase. At least one phase comprises an anesthetic or analgesic (i.e., the phase comprises one or more anesthetics, one or more analgesics, or one or more anesthetics and one or more analgesics).

This invention also is directed, in part, to methods for treating feminine discomfort, dysesthetic vulvodynia, vulvar vestibulitis, or vulvodynia. The methods comprise administering a composition described above.

This invention also is directed, in part, to a use of a composition described above to prepare a medicament. The medicament can be used to treat feminine discomfort, dysesthetic vulvodynia, vulvar vestibulitis, or vulvodynia.

This invention also is directed, in part, to kits for treating feminine discomfort, dysesthetic vulvodynia, vulvar vestibulitis, or vulvodynia. Such kits comprise a composition described above.

Further benefits of Applicants' invention will be apparent to one skilled in the art from reading this patent.

### DETAILED DESCRIPTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent, and may be variously modified.

The compositions of this invention comprise modified-release pharmaceutical compositions. In some embodiments, those compositions comprise extended-release compositions. An extended-release composition generally is a composition that releases at least a substantial portion of one or more active ingredients over an extended period of time following application. In other embodiments, those compositions comprise delayed-release compositions. A delayed-release composition generally is a composition that releases at least a substantial portion of the active ingredient(s) at a time other than promptly after administration.

The modified-release compositions of this invention are generally targeted release compositions. Specifically, they topically deliver at least a substantial portion of the active ingredient(s) to a specific region, organ, or tissue, and, more particularly, to the vaginal cavity surface, vulva surface, and/or skin. As used in this patent, “skin” is the outer covering portion of the body.

The modified-release compositions of this invention are generally bioadherent such that upon topical administration, they generally adhere to the mucous membranes lining the vaginal cavity and the vestibule, to the vulva surface and mucosa, or to the skin; retain their integrity; and/or display physical stability for a long period of time. In some embodiments, the compositions of this invention generally adhere to the mucous membranes lining the vaginal cavity. In some embodiments, the compositions generally adhere to the mucous lining of the vestibule. In some embodiments, the compositions generally adhere to the vulva surface and mucosa. And, in some embodiments, the compositions generally adhere to the skin.

The compositions of this invention comprise a hydrophobic external phase and an aqueous internal phase. The aqueous internal phase is encased or dispersed within the hydrophobic external phase. At least one of the phases comprises an active ingredient (i.e., at least one active ingredient is present in at least one phase). As used in this patent, an “active ingredient” or “drug” is an ingredient responsible for a composition's pharmacologic activity. As discussed below, the active ingredient(s) in the compositions of this invention can be used in the form of salts. Thus, the terms “active ingredient”, “drug”, and “compound” as used in this patent encompass the salts of those active ingredients, drugs, and compounds. For example, “lidocaine” encompasses lidocaine salts (e.g., lidocaine hydrochloride), “diphenhydramine” encompasses diphenhydramine salts (e.g., diphenhydramine hydrochloride), and “anesthetic” encompasses compounds that can be used as anesthetics as well as salts of those compounds that can be used as anesthetics.

The compositions of this invention can comprise an active ingredient in the external phase, internal phase, or both phases. The presence of an active ingredient in a phase depends on, for example, the hydrophobicity or hydrophilicity of the active ingredient, the desired pharmacological profile of the active ingredient, the type of ingredients in the composition, and the like. The desirability of a particular ingredient in a phase depends on, for example, the function of the ingredient, the disease being treated, the environment in which the composition is being applied (e.g., pH), and the like. For example, the compositions of this invention can comprise the same active ingredient (e.g., lidocaine) in more than one phase. In such embodiments, the active ingredient is released from the different phases at different times and/or over different periods of time. For example, a substantial portion of the active ingredient present in one phase can be released promptly after topical administration of the composition while a substantial portion of the active ingredient present in another phase can be released over an extended period of time following application. The compositions of this invention can also comprise two or more active ingredients that are released from the same or different phases at varying times and over varying periods of time. For example, a substantial portion of some active ingredients can be released promptly after topical administration of the composition while a substantial portion of other active ingredients can be released over varying extended periods of time following application.

In some embodiments, an active ingredient is released over at least about 1 minute after topical administration of the composition. In other embodiments, an active ingredient is released over at least about 10 minutes after topical administration of the composition. In yet other embodiments, an active ingredient is released over at least about 3 hours after topical administration of the composition. In yet further embodiments, an active ingredient is released over at least about 6 hours after topical administration of the composition. And in yet further embodiments, an active ingredient is released over at least about 3 days after topical administration of the compositions. Release of the active ingredient(s) will fade at some point after application, which is often no greater than about 10 days after topical administration of the composition.

The compositions of this invention comprise an anesthetic or analgesic. In other words, one or more anesthetics, one or more analgesics, or one or more anesthetics and one or more analgesics may be present in the composition in addition to other ingredients of the composition.

The anesthetic may be, for example, a local anesthetic or topical anesthetic. A “local anesthetic” generally is a drug that suppresses pain perception in a limited body area by local action on sensory nerves. A “topical anesthetic” generally is a local anesthetic that is effective upon application to mucous membranes and/or skin. Suitable anesthetics typically include, for example, ketamine, butamben, pramoxine, dyclonine, etidocaine, benzocaine, dibucaine, cocaine, procaine, prilocaine, chloroprocaine, mepivacaine, bupivacaine, tetracaine, cetacaine, proparacaine, ropivacaine, and lidocaine.

In some embodiments, the anesthetic comprises a topical aesthetic.

In some embodiments, the anesthetic causes little or no irritation upon topical administration to the targeted area.

In some embodiments, the anesthetic has low toxicity upon topical administration to the targeted area.

In some embodiments, the compositions of this invention comprise up to about 10% anesthetic (by weight) (i.e., up to about 10 g anesthetic (total) per about 100 g of composition). In some such embodiments, the compositions comprise from about 0.1 to about 10% anesthetic (by weight). In other such embodiments, the compositions comprise up to about 5% anesthetic (by weight). In yet other such embodiments, the compositions comprise from about 0.5 to about 5% anesthetic (by weight). And, in still further embodiments, the compositions comprise from about 1 to about 3% anesthetic (by weight).

In some embodiments, the anesthetic comprises lidocaine. In some such embodiments, the compositions comprise up to about 10% lidocaine (by weight). In some such embodiments, the compositions comprise from about 0.25 to about 10% lidocaine (by weight). In other such embodiments, the compositions comprise up to about 5% lidocaine (by weight). In yet other such embodiments, the compositions comprise from about 0.5 to about 5% lidocaine (by weight). And, in still further such embodiments, the compositions comprise from about 1 to about 3% lidocaine (by weight).

In some embodiments, the anesthetic is benzocaine. In some such embodiments, the compositions comprise up to about 10% benzocaine (by weight). In some such embodiments, the compositions comprise from about 0.25 to about 10% benzocaine (by weight). In other such embodiments, the compositions comprise up to about 5% benzocaine (by weight). In yet other such embodiments, the compositions comprise from about 0.5 to about 5% benzocaine (by weight). And, in still further such embodiments, the compositions comprise from about 1 to about 3% benzocaine (by weight).

In some embodiments, the anesthetic is tetracaine. In some such embodiments, the compositions comprise up to about 10% tetracaine (by weight). In some such embodiments, the compositions comprise from about 0.25 to about 10% tetracaine (by weight). In other such embodiments, the compositions comprise up to about 5% tetracaine (by weight). In yet other such embodiments, the compositions comprise from about 0.5 to about 5% tetracaine (by weight). And, in still further such embodiments, the compositions comprise from about 1 to about 3% tetracaine (by weight).

In some embodiments, the compositions comprise more than one of pramoxine, benzocaine, dibucaine, tetracaine, cetacaine, dyclonine, and lidocaine.

The analgesic may be, for example, an opioid or non-opioid analgesic.

Suitable opioid analgesics typically include, for example, codeine, dihydrocodeine, fentanyl, butalbital, pentazocine, naloxone, hydrocodone, levorphanol, meperidine, morphine, methadone, oxycodone, butorphanol, oxymorphone, propoxyphene, and meperidine.

Suitable non-opioid analgesics typically include, for example, diclofenac, capsaicin, meprobamate, orphenadrine, methocarbamol, salsalate, carisoprodol, and tramadol.

In some embodiments, the analgesic causes little or no irritation upon topical administration to the targeted area.

In some embodiments, the analgesic has low toxicity upon topical administration to the targeted area.

In some embodiments, the analgesic is effective upon topical administration to the targeted area.

In some embodiments, the compositions of this invention comprise up to about 10% analgesic (by weight) (i.e., up to about 10 g analgesic (total) per about 100 g of composition). In some such embodiments, the compositions comprise from about 0.25 to about 10% analgesic (by weight). In other embodiments, the compositions comprise up to about 5% analgesic (by weight). In yet other embodiments, the compositions comprise from about 0.5 to about 5% analgesic (by weight). And, in still further embodiments, the compositions comprise from about 1 to about 3% analgesic (by weight).

In some embodiments, the analgesic is fentanyl. In some such embodiments, the compositions comprise up to about 10% fentanyl (by weight). In other such embodiments, the compositions comprise from about 0.25 to about 10% fentanyl (by weight). In other such embodiments, the compositions comprise up to about 5% fentanyl (by weight). In yet other such embodiments, the compositions comprise from about 0.5 to about 5% fentanyl (by weight). And in still further such embodiments, the compositions comprise from about 1 to about 3% fentanyl (by weight).

In some embodiments, the analgesic is diclofenac. In some embodiments, the compositions comprise up to about 10% diclofenac (by weight). In some such embodiments, the compositions comprise from about 0.25 to about 10% diclofenac (by weight). In other such embodiments, the compositions comprise up to about 5% diclofenac (by weight). In yet other such embodiments, the compositions comprise from about 0.5 to about 5% diclofenac (by weight). And in still further such embodiments, the compositions comprise from about 1 to about 3% diclofenac (by weight).

In some embodiments, the analgesic is capsaicin. In some embodiments, the compositions comprise up to about 10% capsaicin (by weight). In some such embodiments, the compositions comprise from about 0.01 to about 10% capsaicin (by weight). In other such embodiments, the compositions comprise up to about 5% capsaicin (by weight). In yet other such embodiments, the compositions comprise from about 0.5 to about 5% capsaicin (by weight). And in still further such embodiments, the compositions comprise from about 1 to about 3% capsaicin (by weight).

In some embodiments, the analgesic is tramadol. In some embodiments, the compositions comprise up to about 10% tramadol (by weight). In some such embodiments, the compositions comprise from about 0.25 to about 10% tramadol (by weight). In other such embodiments, the compositions comprise up to about 5% tramadol (by weight). In yet other such embodiments, the compositions comprise from about 0.5 to about 5% tramadol (by weight). And in still further such embodiments, the compositions comprise from about 1 to about 3% tramadol (by weight).

In some embodiments, the total amount of anesthetic(s) and analgesic(s) in the composition is up to about 10% (by weight). In some such embodiments, the total amount of anesthetic(s) and analgesic(s) is from about 0.01 to about 10% (by weight). In other such embodiments, the total amount of anesthetic(s) and analgesic(s) is from about 0.1 to about 10% (by weight). In other embodiments, the total amount of anesthetic(s) and analgesic(s) is up to about 5% (by weight). In yet other embodiments, the total amount of anesthetic(s) and analgesic(s) is from about 0.5 to about 5% (by weight). And in still further embodiments, the total amount of anesthetic(s) and analgesic(s) is from about 1 to about 3% (by weight).

In some embodiments, the compositions of this invention further comprise an immunomodulator (i.e., the compositions comprise one or more immunomodulator drugs). An immunomodulator generally is a drug that weakens or suppresses the immune system, thus decreasing inflammation.

Immunomodulators include, for example, antihistamines. An antihistamine generally is a drug that counteracts the effects of histamine.

Suitable antihistamines for compositions of this invention typically include, for example, H₁ antihistamines, such as, for example, diphenhydramine, chlorpheniramine, hydroxyzine, azelastine, levocabastine, ketotifen, cetirizine, levocetirizine, loratidine, desloratidine, acrivastine, ebastine, fexofenadine, mizolastine, cycloheptadine, azelastine, and promethazine.

Suitable antihistamines for compositions of this invention typically include, for example, H₂ antihistamines, such as, for example, burimamide, cimetidine, ranitidine, famotidine, and nizatidine.

Suitable antihistamines for compositions of this invention typically include, for example, H₃ antihistamines, such as, for example, betahistine, perceptin, ciproxifan, thioperamide, and iodoproxyfan.

In some embodiments, the immunomodulator causes little or no irritation upon topical administration to the targeted area.

In some embodiments, the immunomodulator has low toxicity upon topical administration to the targeted area.

In some embodiments, the immunomodulator is effective upon topical administration to the targeted area.

In some embodiments, the compositions of this invention comprise up to about 10% immunomodulator (by weight) (i.e., up to about 10 g immunomodulator (total) per about 100 g of composition). In some such embodiments, the compositions comprise from about 0.01 to about 10% immunomodulator (by weight). In other embodiments, the compositions comprise up to about 5% immunomodulator (by weight). In yet other embodiments, the compositions comprise from about 0.25 to about 5% immunomodulator (by weight). And in still further embodiments, the compositions comprise from about 1 to about 3% immunomodulator (by weight).

In some embodiments, the compositions comprise an anesthetic, the anesthetic comprises lidocaine, and the immunomodulator comprises diphenhydramine. In some embodiments, the compositions comprise up to about 10% lidocaine (by weight) and up to about 10% diphenhydramine (by weight). In some such embodiments, the compositions comprise from about 0.25 to about 10% lidocaine (by weight), and from about 0.01 to about 10% diphenhydramine (by weight). In other such embodiments, the compositions comprise up to about 5% lidocaine (by weight), and up to about 5% diphenhydramine (by weight). In yet other such embodiments, the compositions comprise from about 1 to about 5% lidocaine (by weight), and from about 0.5 to about 5% diphenhydramine (by weight). And in still further such embodiments, the compositions comprise from about 1 to about 3% lidocaine (by weight), and from about 1 to about 3% diphenhydramine (by weight).

In some embodiments, the compositions of this invention further comprise a cytokine-inhibitory drug (i.e., the compositions comprise one or more cytokine-inhibitory drugs). Cytokines generally act as intercellular signals that mediate reactions between immunoreactive cells. Cytokine-inhibitory drugs generally counteract the effects of cytokines, thus decreasing inflammation. Cytokine-inhibitory drugs may, for example, counteract the effects of interleukin-1, interleukin-4, interleukin-6, tumor necrosis factor-alpha, and/or interferon-gamma.

Cytokine-inhibitory drugs include, for example, antihistamines, such as, for example, the H₁, H₂, and H₃ antihistamines discussed above.

In some embodiments, the cytokine-inhibitory drug causes little or no irritation upon application.

In some embodiments, the cytokine-inhibitory drug has low toxicity upon administration.

In some embodiments, the cytokine-inhibitory drug is effective upon topical administration to mucosa or skin.

In some embodiments, the compositions of this invention comprise up to about 10% cytokine-inhibitory drug (by weight) (i.e., up to about 10 g cytokine-inhibitory drug (total) per about 100 g of composition). In some such embodiments, the compositions comprise from about 0.01 to about 10% cytokine-inhibitory drug (by weight). In other embodiments, the compositions comprise up to about 5% cytokine-inhibitory drug (by weight). In yet other embodiments, the compositions comprise from about 0.25 to about 5% cytokine-inhibitory drug (by weight). And in still further embodiments, the compositions comprise from about 1 to about 3% cytokine-inhibitory drug (by weight).

In some embodiments, the compositions comprise an anesthetic, the anesthetic comprises lidocaine, and the cytokine-inhibitory drug comprises diphenhydramine. In some such embodiments, the compositions comprise up to about 10% lidocaine, and up to about 10% diphenhydramine (by weight). In some such embodiments, the compositions comprise from about 0.25 to about 10% lidocaine (by weight), and from about 0.01 to about 10% diphenhydramine (by weight). In other embodiments, the compositions comprise up to about 5 lidocaine (by weight), and up to about 5% diphenhydramine (by weight). In yet other such embodiments, the compositions comprise from about 1 to about 5% lidocaine (by weight), and from about 0.5 to about 5% diphenhydramine (by weight). And, in still further such embodiments, the compositions comprise from about 1 to about 3% lidocaine (by weight), and from about 1 to about 3% diphenhydramine (by weight).

In some embodiments, the compositions of this invention further comprise an anti-infective drug (i.e., the compositions comprise one or more anti-infective drugs). The anti-infective drug may be, for example, an antibiotic, antifungal, antiviral, antibacterial, or antiprotozoan drug.

Suitable antibiotics typically include, for example, penicillin antibiotics (e.g., amoxicillin, ampicillin, azlocillin, carbenicillin, cloxacillin, dicloxacillin, flucloxacillin, mezlocillin, nafcillin, penicillin, piperacillin, and ticarcillin), aminoglycoside antibiotics (e.g., amikacin, gentamicin, kanamycin, neomycin, netilmicin, streptomycin, and tobramycin), cephalosporin antibiotics (e.g., cefadroxil, cefazolin, cephalexin, cefaclor, cefamandole, cefotetan, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, and cefepime), macrolide antibiotics (e.g., azithromycin, clarithromycin, dirithromycin, erythromycin, and troleandomycin), quinolone antibiotics (e.g., ciprofloxacin, enoxacin, gatifloxacin, levofloxacin, lomefloxacin, moxifloxacin, norfloxacin, ofloxacin, and trovafloxacin), sulfonamide antibiotics (e.g., mafenide, sulfacetamide, sulfamethizole, sulfasalazine, sulfisoxazole, and trimethoprim), tetracyclin antibiotics (e.g., demeclocycline, doxycycline, minocycline, oxytetracycline, and tetracycline), glycopeptide antibiotics (teicoplanin and vancomycin), and polypeptide antibiotics (e.g., bacitracin, colistin, and polymyxin B) as well as chloramphenicol, clindamycin, ethambutol, fosfomycin, furazolidone, isoniazid, linezolid, metronidazole, nitrofurantoin, pyrazinamide, rifampin, and spectinomycin.

Suitable antifungal drugs typically include, for example, butoconazole, fluconazole, and clotrimazole.

Suitable antiviral drugs typically include, for example, valacyclovir, acyclovir, and famciclovir.

A suitable antibacterial drug typically includes, for example, nitrofurantoin.

Suitable antiprotozoan drugs typically include, for example, pentamidine, metronidazole, chloroquine, suramin, trimethoprim, sulfadiazine, albendazole, mebendazole, furazolydone, nitrofurazone, and sulfamethoxazole.

In some embodiments, the compositions of this invention comprise up to about 10% anti-infective drug (i.e., up to about 10 g anti-infective drug (total) per about 100 g of composition). In some such embodiments, the compositions comprise from about 0.001 to about 10% anti-infective drug (by weight). In other embodiments, the compositions comprise up to about 5% anti-infective drug (by weight). In yet other embodiments, the compositions comprise from about 0.25 to about 5% anti-infective drug (by weight). And in still further embodiments, the compositions comprise from about 1 to about 3% anti-infective drug (by weight).

In some embodiments, the composition comprises an anesthetic, the anesthetic comprises lidocaine, and the anti-infective drug comprises an antifungal drug. In some such embodiments, the antifungal drug comprises butoconazole. In some such embodiments, the compositions comprise up to about 10% lidocaine (by weight), and up to about 10% butoconazole (by weight). In other such embodiments, the compositions comprise from about 0.25 to about 10% lidocaine (by weight), and from about 0.01 to about 10% butoconazole (by weight). In yet other such embodiments, the compositions comprise up to about 5% lidocaine (by weight), and up to about 5% butoconazole (by weight). In yet other such embodiments, the compositions comprise from about 1 to about 5% lidocaine (by weight), and from about 0.5 to about 5% butoconazole (by weight). And in still further embodiments, the compositions comprise from about 1 to about 3% lidocaine (by weight), and from about 1% to about 3% butoconazole (by weight).

In some embodiments, the composition comprises an anesthetic, the anesthetic comprises lidocaine, and the anti-infective drug comprises an antibiotic. In some such embodiments, the antibiotic comprises clindamycin. In some such embodiments, the compositions comprise up to about 10% lidocaine (by weight), and up to about 10% clindamycin (by weight). In other such embodiments, the compositions comprise from about 0.25 to about 10% lidocaine (by weight), and from about 0.01 to about 10% clindamycin (by weight). In yet other such embodiments, the compositions comprise up to about 5% lidocaine (by weight), and up to about 5% clindamycin (by weight). In yet further such embodiments, the compositions comprise from about 1 to about 5% lidocaine (by weight), and from about 0.5 to about 5% clindamycin (by weight). And in still further such embodiments, the compositions comprise from about 1 to about 3% lidocaine (by weight), and from about 1 to about 3% clindamycin (by weight).

In some embodiments, the compositions of this invention further comprise an immunomodulator and an anti-infective drug.

In some embodiments, the compositions comprise an anesthetic, the anesthetic comprises lidocaine, the immunomodulator comprises diphenhydramine, and the anti-infective drug comprises butoconazole. In some embodiments, the compositions comprise up to about 10% lidocaine (by weight), up to about 10% diphenhydramine (by weight), and up to about 10% butoconazole (by weight). In some such embodiments, the compositions comprise from about 0.25 to about 10% lidocaine (by weight), from about 0.01 to about 10% diphenhydramine (by weight), and from about 0.01 to about 10% butoconazole (by weight). In other such embodiments, the compositions comprise up to about 5% lidocaine (by weight), up to about 5% diphenhydramine (by weight), and up to about 5% butoconazole (by weight). In yet other such embodiments, the compositions comprise from about 1 to about 5% lidocaine (by weight), from about 0.5 to about 5% diphenhydramine (by weight), and from about 0.5 to about 5% butoconazole (by weight). In still further such embodiments, the compositions comprise from about 1 to about 3% lidocaine (by weight), from about 1 to about 3% diphenhydramine (by weight), and from about 1 to about 3% butoconazole (by weight).

In some embodiments, the compositions comprise an anesthetic, the anesthetic comprises lidocaine, the immunomodulator comprises diphenhydramine, and the anti-infective drug comprises clindamycin. In some embodiments, the compositions comprise up to about 10% lidocaine (by weight), up to about 10% diphenhydramine (by weight), and up to about 10% clindamycin (by weight). In some such embodiments, the compositions comprise from about 0.25 to about 10% lidocaine (by weight), from about 0.01 to about 10% diphenhydramine (by weight), and from about 0.01 to about 10% clindamycin (by weight). In other such embodiments, the compositions comprise up to about 5% lidocaine (by weight), up to about 5% diphenhydramine (by weight), and up to about 5% clindamycin (by weight). In yet other such embodiments, the compositions comprise from about 1 to about 5% lidocaine (by weight), from about 0.5 to about 5% diphenhydramine (by weight), and from about 0.5 to about 5% clindamycin (by weight). And in still further such embodiments, the compositions comprise from about 1 to about 3% lidocaine (by weight), from about 1 to about 3% diphenhydramine (by weight), and from about 1 to about 3% clindamycin (by weight).

In some embodiments, the compositions of this invention further comprise a cytokine-inhibitory drug and an anti-infective drug.

In some embodiments, the compositions comprise an anesthetic, the anesthetic comprises lidocaine, the cytokine-inhibitory drug comprises diphenhydramine, and the anti-infective drug comprises butoconazole. In some embodiments, the compositions comprise up to about 10% lidocaine (by weight), up to about 10% diphenhydramine (by weight), and up to about 10% butoconazole (by weight). In some such embodiments, the compositions comprise from about 0.25 to about 10% lidocaine (by weight), from about 0.01 to about 10% diphenhydramine (by weight), and from about 0.01 to about 10% butoconazole (by weight). In other such embodiments, the compositions comprise up to about 5% lidocaine (by weight), up to about 5% diphenhydramine (by weight), and up to about 5% butoconazole (by weight). In yet other such embodiments, the compositions comprise from about 1 to about 5% lidocaine (by weight), from about 0.5 to about 5% diphenhydramine (by weight), and from about 0.5 to about 5% butoconazole (by weight). And in still further such embodiments, the compositions comprise from about 1 to about 3% lidocaine (by weight), from about 1 to about 3% diphenhydramine (by weight), and from about 1 to about 3% butoconazole (by weight).

In some embodiments, the compositions comprise an anesthetic, the anesthetic comprises lidocaine, the cytokine-inhibitory drug comprises diphenhydramine, and the anti-infective drug comprises clindamycin. In some such embodiments, the compositions comprise up to about 10% lidocaine, up to about 10% diphenhydramine, and up to about 10% clindamycin. In other such embodiments, the compositions comprise from about 0.25% to about 10% lidocaine, from about 0.01% to about 10% diphenhydramine, and from about 0.01% to about 10% clindamycin. In yet other such embodiments, the compositions comprise up to about 5% lidocaine, up to about 5% diphenhydramine, and up to about 5% clindamycin. In yet other such embodiments, the compositions comprise from about 1% to about 5% lidocaine, from about 0.5% to about 5% diphenhydramine, and from about 0.5% to about 5% clindamycin. In yet still further embodiments, the compositions comprise from about 1% to about 3% lidocaine, from about 1% to about 3% diphenhydramine, and from about 1% to about 3% clindamycin.

The compositions of this invention preferably have sufficient viscosity to be bioadherent. In some embodiments, the compositions have a viscosity of up to about 1,200,000 centipoise. In some such embodiments, the compositions have a viscosity of from about 80,000 to about 1,200,000 centipoise. In other such embodiments, the compositions have a viscosity of from about 600,000 to about 1,200,000 centipoise.

In some embodiments, the compositions of this invention have a pH of from about 2 to about 9. In some such embodiments, the compositions have a pH of from about 3.5 to about 7.5. In other such embodiments, the compositions have a pH of about 6 to about 7.

In some embodiments, the osmolarity of the water phase of the compositions of this invention is from about 200 to about 600 milliosmoles/liter. In other embodiments, the osmolarity of the water phase is from about 300 to about 400 milliosmoles/liter.

As discussed above, the compositions of this invention comprise modified-release bioadherent compositions. The compositions generally can be applied in a manner such that they do not seep from the vaginal cavity in an offensive manner, and/or are not easily removed from the surface to which they have been applied. In addition, the compositions generally release at least a substantial portion of the active ingredient(s) (typically in a controlled manner) over an extended period of time (e.g., up to 10 days). As a result, the compositions of this invention generally provide relief comparable or superior to other available treatments, while using smaller amounts of the active ingredient(s) and/or fewer applications. Use of such smaller amounts of active ingredient(s) tends to minimize irritation in the area of application, minimize the amounts of the active ingredient(s) available for absorption into the systemic circulation, and result in overall reduction in exposure to drugs.

The compositions of this invention can be prepared in liquid, semisolid, or solid dosage forms.

In some embodiments, the composition comprises a liquid dosage form comprising an emulsion. An “emulsion” is generally a two-phase system in which one liquid is dispersed throughout another liquid in the form of small droplets. In other embodiments, the composition comprises a liquid dosage form comprising a suspension. A “suspension” generally is a liquid preparation that consists of solid particles dispersed throughout a liquid phase in which the particles are not soluble. In other embodiments, the composition comprises a liquid dosage form comprising a lotion. A “lotion” generally is a fluid suspension or emulsion. In other embodiments, the composition comprises a liquid dosage form comprising a foam. A “foam” generally is an emulsion packaged in a pressurized aerosol container that has a fluffy, semisolid consistency when released after actuating the aerosol valve.

In some embodiments, the composition comprises a semisolid dosage form comprising a cream. A “cream” generally is a semisolid dosage form containing one or more substances dissolved or dispersed in a suitable base. In other embodiments, the composition comprises a semisolid dosage form comprising a gel. A “gel” generally is a semisolid system consisting of either a suspension of small inorganic particles or large organic molecules interpenetrated by a liquid. In other embodiments, the composition comprises a semisolid dosage form comprising an ointment. An “ointment” generally is a semisolid preparation intended for external application to the skin or mucous membrane. In other embodiments, the composition comprises a semisolid dosage form comprising a paste. A “paste” generally is a semisolid dosage form that contains one or more drug substances intended for topical application.

In some embodiments, the composition comprises a solid form comprising, for example, a vaginal suppository or vaginal pessary.

The compositions of this invention generally may be applied to the skin, vaginal cavity surface, and vulva by hand or other means such as, for example, brush, spatula, or other applicator, or by spraying and aerosolization. When the compositions are applied into the vaginal cavity, the patient is preferably in supine position, and the composition is preferably applied high in the vagina.

In some embodiments, a unit dose (i.e., an amount of the composition suitable for a single administration) of any of the compositions of this invention is provided in a disposable, pre-filled applicator.

In some embodiments, the above-described composition is provided in bulk in a suitable container such as, for example, a tube, jar, or package, with a patient or caregiver dispensing the needed dose. In some embodiments, the composition is provided with an applicator that can be used for measuring the needed dose or applying the composition.

The compositions of this invention may be applied multiple times, with periods typically ranging from once per half hour up to once every ten days. In typical embodiments, the compositions are applied once per half hour, once per hour, once per 3 hours, once per 5 hours, once per 8 hours, once per 12 hours, once per day, once per 3 days, once per week, or once per 10 days.

Factors affecting the preferred dosage regimen include the type, age, weight, sex, diet, and condition of the patient; the severity of the pathological condition; the route of administration; pharmacological considerations, such as the activity, efficacy, pharmacokinetic, and toxicology profiles of the particular active ingredient used; whether a drug delivery system is utilized; and whether the active ingredient is administered as part of a drug combination. Thus, the dosage regimen actually employed can vary widely, and, therefore, can deviate from the preferred dosage regimen set forth above.

The compositions of this invention may comprise one or more conventional pharmaceutically acceptable carriers, adjuvants, and/or vehicles (together referred to as “excipients”). Typical excipients may include, for example, cocoa butter; synthetic mono-, di-, or triglycerides; fatty acids; and/or polyethylene glycols. Formulation of drugs is generally discussed in, for example, Hoover, John E., Remington's Pharmaceutical Sciences (Mack Publishing Co., Easton, Pa.: 1975). See also, Liberman, H. A. See also, Lachman, L., eds., Pharmaceutical Dosage Forms (Marcel Decker, New York, N.Y., 1980). Liquid compositions can comprise, for example, wetting, emulsifying, suspending, flavoring (e.g., sweetening), and/or perfuming agents. Suppositories can comprise, for example, a non-irritating excipient that is solid at ordinary temperatures, but liquid at vaginal temperature such that it will melt in the vagina to release the drug.

In some embodiments, the compositions of this invention comprise a permeation enhancer (i.e., the compositions comprise one or more permeation enhancers). A permeation enhancer generally is an agent that facilitates the permeation of a drug through the skin upon topical administration by, for example, reducing the skin's diffusional resistance. Permeation enhancers are selected based on their efficacy in enhancing skin permeation as well as their dermal toxicity and physicochemical and biological compatibility with the active ingredients as well as the rest of the excipients present in a composition. Suitable permeation enhancers include, for example, fatty acids, fatty acid esters, fatty alcohols, fatty acid esters of lactic acid or glycolic acid, glycerol triesters, glycerol diesters, glycerol monoesters, triacetin, short chain alcohols, and dimethyl sulfoxide. Additional permeation enhancers are listed in, for example, Osborne at al., Pharmaceutical Technology 21:50-66 (1997). Methods for assaying the characteristics of permeation enhancers are known in the art. See, for example, Merritt et al., Journal of Controlled Release 1:161-162 (1984).

The active ingredient(s) in the compositions of this invention can be used in the form of salts derived from inorganic or organic acids. Depending on the particular drug, a salt of the drug may be advantageous due to one or more of the salt's physical properties, such as enhanced pharmaceutical stability in differing temperatures and humidities, or a desirable solubility in water or oil.

Where a salt is intended to be administered to a patient (as opposed to, for example, being used in an in vitro context), the salt preferably is pharmaceutically acceptable. Pharmaceutically acceptable salts include salts commonly used to form alkali metal salts and to form addition salts of free acids or free bases. In general, these salts typically may be prepared by conventional means with a compound of this invention by reacting, for example, the appropriate acid or base with the compound.

Pharmaceutically-acceptable acid addition salts of the drugs used in the compositions of this invention may often be prepared from an inorganic or organic acid. Examples of often suitable inorganic acids include hydrochloric, hydrobromic, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Specific examples of often suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), ethanesulfonate, benzenesulfonate, pantothenate, 2-hydroxyethanesulfonate, sulfanilate, cyclohexylaminosulfonate, algenic acid, beta-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Pharmaceutically-acceptable base addition salts of the drugs used in the compositions of this invention include, for example, metallic salts and organic salts. Preferred metallic salts include alkali metal (group Ia) salts, alkaline earth metal (group IIa) salts, and other physiologically acceptable metal salts. Such salts may be made from aluminum, calcium, lithium, magnesium, potassium, sodium, and zinc. Preferred organic salts can be made from amines, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), aralkyl halides (e.g., benzyl and phenethyl bromides), and others.

This invention also is directed, in part, to methods for making the above-described compositions. Compositions of this invention may utilize, for example, the delivery systems described in U.S. Pat. Nos. 4,551,148 and 5,055,303, which are incorporated by reference in their entirety into this patent.

Emulsion compositions of this invention may be prepared by, for example, known batch or continuous processes. As in preparing conventional emulsions, shear force is applied to the components by use of a mixer, homogenizer, mill, impingement surface, ultrasound, shaking, or vibration. Mixing shear should generally be at a relatively low level to prevent destruction of the emulsion by imparting excess energy.

Illustratively, the internal and external phases are prepared separately. As part of a typical batch process, the internal phase is added to the external phase while mixing in a planetary or other suitable type mixer until the emulsion is complete. As part of a typical continuous process, the external phase is introduced into the continuous mixer until it reaches the level of the lowest impeller in the mixing chamber. The two phases are then simultaneously introduced through the bottom of the mixer in proper proportion as the impellers rotate to apply shear to the components. The product emerges through the top of the mixer. Flow rates through the mixing chamber and mixing speed can be adjusted to optimize formation and viscosity.

This invention also is directed, in part, to methods for treating diseases. In this patent, the term “treating” means ameliorating, suppressing, eradicating, preventing, reducing the risk of, and/or delaying the onset of the disease being treated.

In some embodiments, the disease comprises vulvodynia. In other embodiments, the disease comprises vulvar vestibulitis. In other embodiments, the disease comprises dysesthetic vulvodynia. In yet other embodiments, the disease comprises feminine discomfort. Feminine discomfort is characterized by minor or major irritations of the female genitalia (e.g., burning, itching, stinging) that may be aggravated by or induced by, for example, sexual intercourse, menses, or infection.

In some embodiments, the method comprises administering to an animal (typically a mammal) in need of treatment an effective amount of a composition of this invention. In some embodiments, the animal is a human, while in other embodiments, the animal is a mammal other than human. An “effective amount” or “therapeutically-effective amount” means an amount that will achieve the goal of treating the targeted condition.

In some embodiments, the method of this invention comprises a combination therapy wherein a composition of this invention is co-administered with a second (or even a third, fourth, etc.) composition comprising an active ingredient, such as, for example, a fatty acid, an anti-infective drug, an immunomodulator, or cytokine-inhibitory drug. In these embodiments, the composition of this invention and the second composition may be administered in a substantially simultaneous manner (e.g., within about 5 minutes of each other), in a sequential manner, or both. It is contemplated that such combination therapies may include administering one composition multiple times between the administration of the other composition. The time period between the administration of each composition may range from a few seconds (or less) to several hours or days, and will depend on, for example, the properties of each composition and active ingredient (e.g., potency, solubility, bioavailability, half-life, and kinetic profile), as well as the condition of the patient.

The second composition may be administered using a dosage form suitable for the active ingredient(s) present in the second composition to have an intended effect. Contemplated modes of administration for the second composition include, for example, oral, parenteral, inhalation spray, rectal (e.g., suppositories), and topical.

In general, the second composition comprises from about 0.05 to about 95% of an active ingredient(s) (by weight). The preferred composition depends on the method of administration. Such compositions may be prepared by a variety of well-known techniques of pharmacy that include the step of bringing into association the active ingredient(s) with one or more excipients. The compositions are often prepared by uniformly and intimately admixing the active ingredient(s) with a liquid or finely divided solid excipient, and then, if desirable, shaping the product. For example, a tablet may be prepared by compressing or molding a powder or granules of an active ingredient, optionally with one or more excipients and/or one or more other active ingredients. Compressed tablets can be prepared by compressing, in a suitable machine, the therapeutic agent in a free-flowing form, such as a powder or granules optionally mixed with a binder, lubricant, inert diluent and/or surface active/dispersing agent(s). Molded tablets can be made, for example, by molding the powdered compound in a suitable machine. Formulation of drugs is generally discussed in, for example, Hoover, John E., Remington's Pharmaceutical Sciences (Mack Publishing Co., Easton, Pa.: 1975) (incorporated by reference into this patent). See also, Liberman, H. A., Lachman, L., eds., Pharmaceutical Dosage Forms (Marcel Decker, New York, N.Y., 1980) (incorporated by reference into this patent). See also, Kibbe et al., eds., Handbook of Pharmaceutical Excipients, 3rd Ed., (American Pharmaceutical Association, Washington, D.C. 1999) (incorporated by reference into this patent).

Active ingredients suitable for oral administration may be administered in discrete units comprising, for example, solid dosage forms. Such solid dosage forms include, for example, hard or soft capsules, cachets, lozenges, tablets, pills, powders, or granules, each containing a pre-determined amount of the active ingredient(s). In such solid dosage forms, the active ingredient(s) is ordinarily combined with one or more excipients. If administered per os, the active ingredient(s) may be mixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Pharmaceutical compositions particularly suitable for buccal (sub-lingual) administration include, for example, lozenges comprising the active ingredient(s) in a flavored base, usually sucrose, and acacia or tragacanth; or pastilles comprising the active ingredient(s) in an inert base, such as gelatin and glycerin or sucrose and acacia.

Active ingredients suitable for oral administration also can be administered in discrete units comprising, for example, liquid dosage forms. Such liquid dosage forms include, for example, pharmaceutically acceptable emulsions (including both oil-in-water and water-in-oil emulsions), solutions (including both aqueous and non-aqueous solutions), suspensions (including both aqueous and non-aqueous suspensions), syrups, and elixirs containing inert diluents commonly used in the art (e.g., water). Such compositions also may comprise excipients, such as wetting, emulsifying, suspending, flavoring (e.g., sweetening), and/or perfuming agents.

Oral delivery of the therapeutic agents in the present invention may include formulations that provide immediate delivery, or, alternatively, extended or delayed delivery of the active ingredient(s) by a variety of mechanisms. Immediate delivery formulations include, for example, oral solutions, oral suspensions, fast-dissolving tablets or capsules, disintegrating tablets, etc. Extended or delayed delivery formulations include, for example, pH-sensitive release from the dosage form based on the changing pH of the gastrointestinal tract, slow erosion of a tablet or capsule, retention in the stomach based on the physical properties of the formulation, bio-adhesion of the dosage form to the mucosal lining of the intestinal tract, or enzymatic release of the active drug from the dosage form. The intended effect is to extend the time period over which the active drug molecule is delivered to the site of action by manipulation of the dosage form. Thus, in the case of capsules, tablets, and pills, the dosage forms may comprise buffering agents, such as sodium citrate, or magnesium or calcium carbonate or bicarbonate. Tablets and pills additionally may be prepared with enteric coatings. Suitable enteric coatings include, for example, cellulose acetate phthalate, polyvinylacetate phthalate, hydroxypropylmethyl-cellulose phthalate, and anionic polymers of methacrylic acid and methacrylic acid methyl ester.

“Parenteral administration” includes subcutaneous injections, intravenous injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents. Acceptable excipients typically include, for example, water, 1,3-butanediol, Ringer's solution, isotonic sodium chloride solution, bland fixed oils (e.g., synthetic mono- or diglycerides), dextrose, mannitol, fatty acids (e.g., oleic acid), dimethyl acetamide, surfactants (e.g., ionic and non-ionic detergents), and/or polyethylene glycols (e.g., PEG 400).

Formulations for parenteral administration may, for example, be prepared from sterile powders or granules having one or more of the excipients mentioned for use in the formulations for oral administration. The active ingredient(s) may be dissolved in water, polyethylene glycol, propylene glycol, ethanol, corn oil, cottonseed oil, peanut oil, sesame oil, benzyl alcohol, sodium chloride, and/or various buffers. The pH may be adjusted, if necessary, with a suitable acid, base, or buffer.

Other excipients and modes of administration known in the pharmaceutical art also may be used.

In some combination therapies, the second composition comprises a fatty acid. Such a fatty acid may be, for example, an essential fatty acid such as, for example, an omega-3 or omega-6 fatty acid. Omega-6 essential fatty acids include, for example, linoleic acid and arachidonic acid. Omega-3 essential fatty acids include, for example, alpha-linolenic acid, eicosapentaenoic acid, and docosahexaenoic acid.

An essential fatty acid may be used in the form of various derivatives, for example, salts of inorganic and organic acids as described above, phospolipid esters, ethers, and sterol derivatives. Linoleic acid can be used as, for example, phosphatidal choline esters, phosphatidal ether, and sipolsterol ester. Linolenic acid can be used as, for example, phosphatidal choline esters, phosphatidal ether, and sipolsterol ester.

The essential fatty acids may generally be from a variety of sources, such as, for example, natural or synthetic oils, fats, waxes, and mixtures thereof. They may be derived from, for example, partially hydrogenated oils, non-hydrogenated oils, and fully hydrogenated oils. Illustrative sources of essential fatty acids include seed oil, fish oil, marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, tallow, beef tallow, butter, chicken fat, lard, dairy butterfat, and shea butter.

In some embodiments, the second composition comprises an essential fatty acid composition comprising linoleic acid, linolenic acid, or docosahexaenoic acid. In some such embodiments, for example, the essential fatty acid composition comprises linoleic acid, linolenic acid, and docosahexaenoic acid, and the ratio of the sum of the amounts of linoleic acid and linolenic acid to the amount of docosahexaenoic acid is from about 1:0.5 to about 1:1.5.

In some embodiments, the second composition comprises an essential fatty acid composition described in U.S. Pat. No. 6,479,545 (incorporated by reference into this patent).

A fatty acid composition may be administered by any means that produces contact of fatty acid with its intended target. As discussed above, the essential fatty acids can be administered as, for example, a compound per se or a pharmaceutically-acceptable salt thereof. Pharmaceutically-acceptable salts are often particularly suitable for medical applications because of their greater aqueous solubility relative to the compounds themselves. Often, a fatty acid composition is preferably administered orally. This invention, however, also contemplates methods wherein a fatty acid is administered by another means, such as parenterally.

In many embodiments, a fatty acid composition is administered as part of a pharmaceutical composition that further comprises a pharmaceutically-acceptable excipient or another active ingredient. Typical oral dosage forms comprising a fatty acid comprise up to about 4000 mg essential fatty acid(s), and particularly from about 10 mg to about 4000 mg essential fatty acid(s).

This invention also is directed, in part, to a use of an anesthetic- and/or analgesic-comprising composition of this invention to prepare a medicament that is used to treat a disease in an animal. In some embodiments, the animal is a mammal. In some such embodiments, the mammal is a human. In some embodiments, the disease comprises feminine discomfort. In other embodiments, the disease comprises vulvodynia. In other embodiments, the disease comprises dysesthetic vulvodynia. In other embodiments, the disease comprises vulvar vestibulitis.

This invention also is directed, in part, to a kit comprising an anesthetic- and/or analgesic-comprising composition of this invention. The kit is used to treat a disease in an animal. In some embodiments, the animal is a mammal. In some such embodiments, the mammal is a human. In some embodiments, the disease comprises feminine discomfort. In other embodiments, the disease comprises vulvodynia. In other embodiments, the disease comprises dysesthetic vulvodynia. In other embodiments, the disease comprises vulvar vestibulitis.

In some embodiments, a composition of this invention is provided in the kit in a disposable, pre-filled applicator. In other embodiments, the composition is provided in the kit in bulk in a suitable container such as, for example, a tube, jar, or package. In some such embodiments, the composition is provided with a means for measuring or applying the composition. In some such embodiments, the composition is provided with an applicator that can be used for measuring the needed dose and applying the composition. In some such embodiments, the applicator is a disposable one.

In some embodiments, the kit further comprises instructions for, for example, using the kit.

In some embodiments, the kit further comprises a means for applying the composition, such as, for example, a brush, spatula, or other applicator. In some such embodiments, the means for applying the composition (e.g., an applicator) can also be used to measure the needed dose.

In some embodiments, the kit further comprises a second (or even a third, fourth, etc.) composition comprising an active ingredient, such as, for example, a fatty acid, anti-infective drug, immunomodulator drug, or cytokine-inhibitory drug. In some such embodiments, the second composition comprises an oral dosage form. In other such embodiments, the second composition comprises a parental dosage form.

### EXAMPLES

The following examples are merely illustrative, and not limiting to this disclosure in any way.

### Example 1

### Development and Characterization of Prototype Compositions

Development and in vitro characterization of prototype compositions is conducted to identify optimal compositions for use in clinical studies. In vitro experiments are conducted to characterize the release characteristics of the active ingredients and to assess the stability of the prototype compositions. Compositions with known release characteristics and stability as well as compositions without any active ingredients (i.e., placebo compositions) are tested as controls. Stability and microbiology challenge test are conducted as well.

For example, prototype formulations comprising 5, 4, 3, 2, and 1% lidocaine, 2% diphenhydramine, and various combinations of lidocaine and diphenhydramine are developed and tested via the watch glass method to assess the release characteristics of those compositions. Formal stability and microbiology challenge tests of those compositions are performed as well. Placebo prototype compositions are developed and characterized as well.

### Example 2

### Human Bioavailability Study

One or more human bioavailability studies are conducted to obtain information about the absorption of active ingredients, to determine if there is correlation between in vitro release characteristics and bioavailability, and to provide safety information about potential active ingredients' toxicity.

A bioavailability study can be conducted for compositions comprising one or more active ingredients. For example, for the development of compositions for treating vulvodynia, a single-dose, parallel group study in healthy women is conducted with prototype compositions comprising 5, 3, and 1% lidocaine. Blood is drawn about 15-20 times, and an evaluation of how long the compositions are visually present in the vaginal vault is conducted.

A bioavailability study can be conducted for compositions comprising more than one active ingredients. For example, for the development of compositions for treating vulvodynia, a single-dose, parallel group study in healthy women is conducted with three or four compositions, including lidocaine, diphenhydramine, and lidocaine/diphenhydramine combination composition. The lidocaine composition may contain the highest dose of lidocaine that was not irritating in an animal irritation study and that would not result in systemic absorption above the potential toxicity levels. A lidocaine/diphenhydramine combination composition with a lower dose of lidocaine may also be tested. Blood is drawn about 15-20 times, and an evaluation of how long the compositions are visually present in the vaginal vault is conducted.

### Example 3

### Animal Irritation Study

Animal irritation study is conducted to determine the highest tolerated concentrations of active ingredients in, for example, rabbits. An animal irritation study may be conducted before of after bioavailability study results for the active ingredients are available.

For example, rabbit irritation studies are conducted with a total of seven prototype compositions: two lidocaine compositions (for example, one composition containing the lowest dose of lidocaine resulting in systemic concentrations of lidocaine, and a second composition containing the highest dose of lidocaine not resulting in systemic concentrations of lidocaine); one diphenhydramine composition (containing 2% diphenhydramine); two lidocaine/diphenhydramine combination compositions; a placebo composition; and a sham composition.

### Example 4

### Illustrative Compositions

The following compositions were prepared utilizing the methods described above.

| | |
|---|---|
| Ingredient Name | Weight (%) |

Composition 1

| | |
|---|---|
| Purified Water, USP | 40.43 |
| Edetate Disodium, USP | 0.05 |
| Sorbitol Solution, USP | 35.00 |
| Diphenhydramine HCl, USP | 2.00 |
| Lidocaine HCl, USP | 5.00 |
| Mineral Oil, USP | 8.46 |
| Polyglycerol-3 Oleate | 2.70 |
| Glyceryl Monoisostearate | 2.70 |
| Egg Yolk Lecithin | 2.00 |
| Hydrophobic Silicon Dioxide | 1.01 |
| Microcrystalline Wax, NF | 0.40 |
| Methylparaben, NF | 0.20 |
| Propylparaben, NF | 0.05 |
| | 100.00 |

Composition 2

| | |
|---|---|
| Purified Water, USP | 42.43 |
| Edetate Disodium, USP | 0.05 |
| Sorbitol Solution, USP | 34.40 |
| Diphenhydramine HCl, USP | 2.00 |
| Lidocaine HCl, USP | 5.00 |
| Mineral Oil, USP | 8.46 |
| PEG 30 Dipolyhydroxystearate | 4.00 |
| Glyceryl Monoisostearate | 2.00 |
| Hydrophobic Silicon Dioxide | 1.01 |
| Microcrystalline Wax, NF | 0.40 |
| Methylparaben, NF | 0.20 |
| Propylparaben, NF | 0.05 |
| | 100.00 |

Composition 3

| | |
|---|---|
| Purified Water, USP | 41.43 |
| Edetate Disodium, USP | 0.05 |
| Sorbitol Solution, USP | 34.40 |
| Diphenhydramine HCl, USP | 2.00 |
| Lidocaine HCl, USP | 5.00 |
| Mineral Oil, USP | 8.46 |
| PEG 30 Dipolyhydroxystearate | 4.00 |
| Glyceryl Monoisostearate | 2.00 |
| Egg Yolk Lecithin | 1.00 |
| Hydrophobic Silicon Dioxide | 1.01 |
| Microcrystalline Wax, NF | 0.40 |
| Methylparaben, NF | 0.20 |
| Propylparaben, NF | 0.05 |
| | 100.00 |

Composition 4

| | |
|---|---|
| Purified Water, USP | 41.43 |
| Edetate Disodium, USP | 0.05 |
| Sorbitol Solution, USP | 34.40 |
| Diphenhydramine HCl, USP | 2.00 |
| Lidocaine HCl, USP | 5.00 |
| Mineral Oil, USP | 8.46 |
| PEG 30 Dipolyhydroxystearate | 2.00 |
| Glyceryl Monoisostearate | 2.00 |
| Egg Yolk Lecithin | 3.00 |
| Hydrophobic Silicon Dioxide | 1.01 |
| Microcrystalline Wax, NF | 0.40 |
| Methylparaben, NF | 0.20 |
| Propylparaben, NF | 0.05 |
| | 100.00 |

All references cited above are incorporated by reference into this patent. The discussion of those references is intended merely to summarize the assertions made by their authors. No admission is made that any reference (or a portion of any reference) is relevant prior art. Applicants reserve the right to challenge the accuracy and pertinence of the cited references.

The words “comprise”, “comprises”, and “comprising” are to be interpreted inclusively rather than exclusively.

1. A modified-release pharmaceutical composition, wherein:
the composition is bioadherent to a vaginal cavity surface, vulva surface, or skin; and
the composition comprises:
a hydrophobic external phase, and
an aqueous internal phase encased or dispersed within the external phase; and
at least one of the phases comprises a drug selected from the group consisting of an anesthetic and analgesic.

2. The composition of claim 1, wherein the composition comprises more than one anesthetic or analgesic.

3. The composition of claim 1, wherein the composition comprises an anesthetic selected from the group consisting of lidocaine, ketamine, butamben, pramoxine, dyclonine, etidocaine, benzocaine, dibucaine, cocaine, procaine, prilocaine, chloroprocaine, mepivacaine, bupivacaine, tetracaine, cetacaine, proparacaine, and ropivacaine.

4. The composition of claim 3, wherein the composition comprises lidocaine hydrochloride.

5. The composition of claim 1, wherein the composition comprises from about 0.25 to about 10% lidocaine (by weight).

6. The composition of claim 1, wherein the composition comprises from about 0.5 to about 5% lidocaine (by weight).

7. The composition of claim 1, wherein the composition comprises an analgesic selected from the group consisting of codeine, dihydrocodeine, fentanyl, butalbital, pentazocine, naloxone, hydrocodone, levorphanol, meperidine, morphine, methadone, oxycodone, butorphanol, oxymorphone, propoxyphene, diclofenac, capsaicin, meprobamate, orphenadrine, methocarbamol, salsalate, carisoprodol, and tramadol.

8. The composition of claim 1, wherein the composition further comprises an immunomodulator drug.

9. The composition of claim 8, wherein the composition comprises more than one immunomodulator drug.

10. The composition of claim 8, wherein the immunomodulator drug comprises an antihistamine.

11. The composition of claim 8, wherein the immunomodulator drug comprises a drug selected from the group consisting of diphenhydramine, chlorpheniramine, hydroxyzine, azelastine, levocabastine, ketotifen, cetirizine, levocetirizine, loratidine, desloratidine, acrivastine, ebastine, fexofenadine, mizolastine, cycloheptadine, azelastine, promethazine, burimamide, cimetidine, ranitidine, famotidine, and nizatidine, betahistine, perceptin, ciproxifan, thioperamide, and iodoproxyfan.

12. The composition of claim 11, wherein the immunomodulator drug comprises diphenhydramine hydrochloride.

13. The composition of claim 8, wherein:
the composition comprises lidocaine, and
the immunomodulator drug comprises diphenhydramine.

14. The composition of claim 8, wherein the composition comprises:
from about 0.25 to about 10% lidocaine (by weight), and
from about 0.01 to about 10% diphenhydramine (by weight).

15. The composition of claim 8, wherein the composition comprises:
from about 0.5 to about 5% lidocaine (by weight), and
from about 1 to about 3% diphenhydramine (by weight).

16. The composition of claim 1, wherein the composition further comprises a cytokine-inhibitory drug.

17. The composition of claim 16, wherein the composition comprises more than one cytokine-inhibitory drug.

18. The composition of claim 16, wherein the cytokine-inhibitory drug comprises an antihistamine.

19. The composition of claim 16, wherein the cytokine-inhibitory drug comprises a drug selected from the group consisting of diphenhydramine, chlorpheniramine, hydroxyzine, azelastine, levocabastine, ketotifen, cetirizine, levocetirizine, loratidine, desloratidine, acrivastine, ebastine, fexofenadine, mizolastine, cycloheptadine, azelastine, promethazine, burimamide, cimetidine, ranitidine, famotidine, and nizatidine, betahistine, perceptin, ciproxifan, thioperamide, and iodoproxyfan.

20. The composition of claim 19, wherein the cytokine-inhibitory drug comprises diphenhydramine hydrochloride.

21. The composition of claim 16, wherein:
the composition comprises lidocaine, and
the cytokine-inhibitory drug comprises diphenhydramine.

22. The composition of claim 16, wherein the composition comprises:
from about 0.25 to about 10% lidocaine (by weight), and
from about 0.01 to about 10% diphenhydramine (by weight).

23. The composition of claim 16, wherein the composition comprises:
from about 0.5 to about 5% lidocaine (by weight), and
from about 1 to about 3% diphenhydramine (by weight).

24. The composition of claim 1, wherein the composition further comprises an anti-infective drug.

25. The composition of claim 24, wherein the composition comprises more than one anti-infective drug.

26. The composition of claim 0, wherein the anti-infective drug comprises an antifungal drug.

27. The composition of claim 24, wherein the anti-infective drug comprises a drug selected from the group consisting of fluconazole and clotrimazole.

28. The composition of claim 24, wherein the anti-infective drug comprises butoconazole.

29. The composition of claim 24, wherein:
the composition comprises lidocaine, and
the anti-infective drug comprises butoconazole.

30. The composition of claim 24, wherein the composition comprises:
from about 0.25 to about 10% lidocaine (by weight), and
from about 0.01 to about 10% butoconazole (by weight).

31. The composition of claim 24, wherein the composition comprises:
from about 0.5 to about 5% lidocaine (by weight), and
from about 1 to about 3% butoconazole (by weight).

32. The composition of claim 24, wherein the anti-infective drug comprises an antibiotic.

33. The composition of claim 24, wherein the anti-infective drug comprises an antibiotic selected from the group consisting of a penicillin antibiotic, aminoglycoside antibiotic, cephalosporin antibiotic, macrolide antibiotic, quinolone antibiotic, sulfonamide antibiotic, tetracyclin antibiotic, glycopeptide antibiotic, and popypeptide antibiotic.

34. The composition of claim 24, wherein the anti-infective drug comprises an antibiotic selected from the group consisting of gentamicin, cefaclor, cefotetan, cefuroxime, cefadroxil, nitrofurantoin, and demeclocycline.

35. The composition of claim 24, wherein the anti-infective drug comprises clindamycin.

36. The composition of claim 24, wherein:
the composition comprises lidocaine, and
the anti-infective drug comprises clindamycin.

37. The composition of claim 24, wherein the composition comprises:
from about 0.25 to about 10% lidocaine (by weight), and
from about 0.1 to about 10% clindamycin (by weight).

38. The composition of claim 24, wherein the composition comprises:
from about 0.5 to about 5% lidocaine (by weight), and
from about 1 to about 3% clindamycin (by weight).

39. The composition of claim 1, wherein the composition is bioadherent to a mammalian vaginal cavity surface, vulva surface, or skin.

40. The composition of claim 0, wherein the composition is bioadherent to a mammalian vaginal cavity surface, vulva surface, and skin.

41. The composition of claim 1, wherein the composition releases the drug(s) over at least about 3 hours upon topical administration to a mammalian vaginal cavity surface, vulva surface, or skin.

42. The composition of claim 1, wherein the composition releases the drug(s) over at least about 1 minute upon topical administration to a mammalian vaginal cavity surface, vulva surface, or skin.

43. The composition of claim 1, wherein the composition comprises a liquid or a semi-solid with a viscosity of from about 80,000 to about 1,200,000 centipoise.

44. The composition of claim 1, wherein the composition has a pH of from about 2 to about 9.

45. The composition of claim 0, wherein the composition has a pH of from about 3.5 to about 7.5.

46. The composition of claim 0, wherein the composition has a pH of from about 6 to about 7.

47. The composition of claim 1, wherein the composition comprises a dosage form selected from the group consisting of cream, emulsion, gel, lotion, ointment, paste, suspension, and suppository.

48. The composition of claim 1, wherein the composition further comprises a permeation enhancer.

49. A method for treating a disease selected from the group consisting of feminine discomfort, dysesthetic vulvodynia, vulvar vestibulitis, and vulvodynia in a mammal in need of such treatment, wherein the method comprises administering to the mammal an effective amount of the composition of claim 1.

50. The method of claim 49, wherein the disease comprises feminine discomfort.

51. The method of claim 49, wherein the disease comprises dysesthetic vulvodynia.

52. The method of claim 49, wherein the disease comprises vulvodynia.

53. The method of claim 49, wherein the disease comprises vulvar vestibulitis.

54. The method of claim 49, wherein the method further comprises administering to the mammal a second composition comprising a compound selected from the group consisting of a fatty acid, anti-infective drug, immunomodulator drug, and cytokine-inhibitory drug.

55. The method of claim 54, wherein the second composition comprises a fatty acid.

56. The method of claim 55, wherein the second composition comprises more than one fatty acid.

57. The method of claim 54, wherein the composition of any one of claims 0-0 and the second composition are administered in a substantially simultaneous manner.

58. The method of claim 54, wherein the second composition is administered orally.

59. The method of claim 54, wherein the second composition is administered parenterally.

60-61. (canceled)

62. A kit for treating a disease selected from the group consisting of feminine discomfort, dysesthetic vulvodynia, vulvar vestibulitis, and vulvodynia in a mammal in need of such treatment, wherein the kit comprises an effective amount of a composition of claim 1.

63. The kit of claim 62, wherein the kit further comprises an applicator.

64. The kit of claim 62, wherein the kit further comprises a second composition comprising a compound selected from the group consisting of fatty acid, anti-infective drug, immunomodulator, and cytokine-inhibitory drug.

65. The kit of claim 64, wherein the second composition comprises an oral dosage form.

66. The kit of claim 64, wherein the second composition comprises a parenteral dosage form.
